(19) United States
(12) Patent Application Publication
(10) Pub. No.: US 2022/0338520 A1
(43) Pub. Date: Oct. 27, 2022

(54) FOOD COMPOSITIONS FOR LACTATING WOMEN

(71) Applicant: SOCIETE DES PRODUITS NESTLE S.A., Vevey (CH)

(72) Inventors: MATHILDE FLEITH, Saint-Legier (CH); COLLEEN FOGARTY DRAPER, St Sulpice (CH); TINU MARY SAMUEL, La Tour De Peilz (CH); DANTONG WANG, Prilly (CH)

(21) Appl. No.: 17/640,600

(22) PCT Filed: Sep. 3, 2020
(86) PCT No.: PCT/EP2020/074564

§ 371 Date: Mar. 4, 2022

(30) Foreign Application Priority Data
Sep. 5, 2019 (EP) 19195657.2

(51) Int. Cl. A23L 33/155; A23L 33/00

(52) U.S. Cl. CPC A23L 33/155; A23L 33/40; A23V 2250/702; A23V 2250/71; A23V 2200/30

(57) ABSTRACT

The present invention concerns a food composition for use in preventing nutrients' inadequacies in lactating women with high fiber type dietary patter.

### FIELD OF THE INVENTION

The present invention concerns a food composition to compensate nutrition inadequacies. In particular, it relates to a food composition useful to prevent dietary inadequacies in lactating women having a high fiber type dietary intake pattern.

### BACKGROUND OF THE INVENTION

Lactation is an important life stage, it brings additional nutritional challenges to lactating women. EFSA provides special nutrient intake recommendations for lactating women to address the increased requirements.

Daily food intakes determine the daily dietary nutrient intakes. People's long term dietary habits, which can be reflected in dietary patterns for different groups of people, determine their nutritional status. Thus the nutritional needs can be assessed based on their dietary patterns. Currently, there is no report on the eating habits nor identified dietary patterns in lactating women. Identifying and understanding dietary patterns in lactating women would eallow to provide recommendations and tailor-made services and products to tackle the nutritional needs linked to each dietary pattern.

Accordingly, it is an object of the present invention to provide a food composition preventing nutrients inadequacies in lactating women having defined eating habits and associated food intake dietary patterns.

### SUMMARY OF THE INVENTION

The present invention provides a solution to the above mentioned problem as set out in the claims.

### BRIEF DESCRIPTION OF THE DRAWINGS

FIGS. 1 to 4 schematically represent the food intake features of each cluster of dietary pattern identified as described in Example 1 (Clusters 1 to 4, respectively). X-axis shows the z score of the difference between individual intake of of people each group (each “cluster”) and the mean intake of total population. Y-axis shows the food groups.

### DETAILED DESCRIPTION OF THE INVENTION

### Definitions

Prior to discussing the present invention in further details, the following terms and conventions will first be defined.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 1 to 10 (including 1 and 10), from 2 to 8, from 3 to 7, from 1 to 9, from 3.6 to 4.6, from 3.5 to 9.9, and so forth. All references to singular characteristics or limitations of the present invention shall include the corresponding plural characteristic or limitation, and vice versa, unless otherwise specified or clearly implied to the contrary by the context in which the reference, is made.

The term “and/or” used in the context of the “X and/or Y” should be interpreted as “X”, or “Y”, or “X and Y”.

Unless defined otherwise, all technical and scientific terms used herein have the same meaning as commonly understood by one of ordinary skilled in the art.

Within the context of the present invention, the expression “having high fiber type dietary pattern” is to be understood as referred to a subject, specifically a lactating woman, whose daily dietary intake for food groups according to the 2013 version of the French food composition database of the Centre d'information sur la qualité des aliments (Ciqual) (France) falls within the following ranges:

Range

| Food intake (grams/day) | From | To |
|---|---|---|
| Alc_beverages | 0.0 | 111.4 |
| Breads_and_rolls | 20.5 | 149.9 |
| Brk_cereals_and_cereal_bar | 0.0 | 65.1 |
| Cereals_and_pasta | 24.5 | 158.7 |
| Cheeses | 8.6 | 69.9 |
| Eggs_and_egg_products | 0.0 | 34.8 |
| Fats_and_oils | 9.5 | 35.6 |
| Fish_and_bat | 0.0 | 53.8 |
| Fruits | 55.5 | 370.1 |
| Juices_and_nectars | 0.0 | 166.7 |
| Legumes | 0.0 | 33.3 |
| Meat_products | 0.0 | 58.9 |
| Milk_and_milk_products | 50.1 | 469.4 |
| Mixed_dishes | 22.8 | 153.9 |
| Mixed_salads | 0.0 | 11.1 |
| Nuts_and_seeds | 0.0 | 36.5 |
| Pastry_and_biscuits | 11.3 | 80.9 |
| Poultry | 0.0 | 52.7 |
| Red_meat | 0.0 | 105.8 |
| Sandwiches | 0.0 | 0.0 |
| Seasonings_and_sauces | 5.4 | 49.4 |
| Shellfish_and_mol | 0.0 | 26.6 |
| Soups_and_stocks | 0.0 | 94.2 |
| Starchy_tubers | 2.6 | 105.4 |
| Coffee_tea_cocoa_beverages | 0.0 | 489.3 |
| Prepa_for_non_Alc_beverages | 0.0 | 0.0 |
| Soft_drinks | 0.0 | 170.7 |
| Sugars_and_confectionery | 4.1 | 38.0 |
| Vegetables | 92.3 | 337.4 |

Within the context of the present invention, the term “nutrient” or nutrients” is intended to comprise both macronutrients (for example carbohydrates, proteins or fats) and micronutrients (for example minerals or vitamins) for the human body.

Within the context of the present invention, the terms “ingredient” or “ingredients” indicate an edible substance or mixture of substances which comprise or is essentially consisting of a nutrient for the human body.

In one embodiment of the present invention, the terms “ingredient” or “ingredients” indicate an edible substance essentially consisting of a nutrient for the human body.

Within the context of the present invention, the term “ingredient X” or “(nutrient) X” indicates an edible substance or mixture of substances which comprise or is essentially consisting of at least one substance capable of delivering the specified nutrient X to the human body.

In one embodiment, the term “ingredient X” or “(nutrient) X” indicates an edible substance essentially consisting of substance capable of delivering the specified nutrient X to the human body.

Within the context of the present invention, the terms “ingredient providing nutrient X” or “ingredients providing nutrient X” indicate an edible substance and/or mixture of substances which comprise or is essentially consisting of at least one substance capable of delivering the specified nutrient X to the human body.

Within the context of the present invention, the terms “ingredient providing nutrient X in amount Y” or “ingredients providing nutrient X in amount Y” indicate an edible substance and/or mixture of substances which comprise or is essentially consisting of at least one substance capable of delivering the specified nutrient X to the human body in the specified amount Y.

The expressions “fiber” or “fibers” or “dietary fiber” or “dietary fibers” within the context of the present invention indicate the indigestible portion, in small intestine, of food derived from plants which comprises two main components: Soluble fiber, which dissolves in water, and insoluble fiber. Mixtures of fibers are comprised within the scope of the terms above mentioned. Soluble fiber is readily fermented in the colon into gases and physiologically active byproducts, and can be prebiotic and viscous. Insoluble fiber does not dissolve in water, is metabolically inert and provides bulking, or it can be prebiotic and metabolically ferment in the large intestine. Chemically, dietary fiber consists of (1) non-starch polysaccharides (NSP) such as arabinoxylans, cellulose, beta-glucans and many other plant components such as inulin, pectins or gums; (2) resistant oligosaccharides such as resistant dextrins; (3) resistant starch and (4) associated substances from plants such as lignin or chitins. Non limiting examples of dietary fibers are: prebiotic fibers such as Fructo-oligosaccharides (FOS), inulin, galacto-oligosaccharides (GOS), fruit fiber, legume fiber, vegetable fiber, cereal fiber, resistant starch such as high amylose corn starch. As fibers are not digestible, they do not contain available carbohydrates.

The total fiber content of the food composition is provided by the sum of amount of fiber naturally present in all the ingredients used in the recipe (for example from whole grain cereal flour, from fruit or vegetables).

As it will be apparent to the skilled person, grain products, Bread, Breakfast cereals, potatoes, vegetables, legumes and fruit according to the present invention may bring certain amount of fibers to the food composition of the present invention.

Within the context of the present invention, the term “nutrients' inadequacy” or “dietary inadequacy” indicates that the total daily dietary intake of a nutrient in a certain lactating subject is below the EFSA′ daily intake recommendation for lactating women and/or below well established nutritional requirement for lactation.

Within the context of the present invention, the expression “prevent nutrients' inadequacy” should be understood to include prevention of nutrients' inadequacies as well as reduction of the risk of nutrients' inadequacies in lactating women.

The composition of the present invention, including the many embodiments described herein, can comprise, consist of, or consist essentially of the essential elements and limitations of the invention described herein, as well as any additional or optional ingredients, components, or limitations described herein.

Food Composition

In one embodiment, the food composition according to the present invention comprises Vitamin A and Vitamin D.

In one embodiment, the food composition according to the present invention is of synthetic origin, e.g. prepared by admixing ingredients and optionally excipients.

In one embodiment, the food composition according to the present invention comprises
Vitamin A in an amount of at least 350 μg per serving;
Vitamin D in an amount of at least 9 μg per serving;

Wherein the food composition is intended for a single daily administration.

In another embodiment, the food composition according to the present invention comprises:
Vitamin A in an amount ranging from 350 μg to 3000 μg per serving;
Vitamin D in an amount ranging from 9 μg to 100 μg per serving;

Wherein the food composition is intended for a single daily administration.

In another embodiment, the food composition according to the present invention comprises:
Vitamin A in an amount ranging from 350 μg to 600 μg per serving;
Vitamin D in an amount ranging from 9 μg to 20 μg per serving;

Wherein the food composition is intended for a single daily administration.

Food compositions according to the present invention may comprise ingredients which are sources of additional nutrient such as proteins, fats, carbohydrates, prebiotics, probiotics, other vitamins and/or minerals. The maternal food composition may have any form that is accepted by women as part of their diet or as nutritional supplement.

For example, the food composition according to the present invention may be selected from the group consisting of a powdered nutritional composition to be reconstituted in milk or water, a powdered nutritional composition stored in a single use capsule or pod to be reconstituted in milk or water, a nutritional formula, a cereal based-product, a drink, a bar, a nutritional supplement, a pre-mix (for example of minerals and vitamins) for fortification purposes, a nutraceutical, a yogurt, a dairy based product, a food sprinkler, a gummy, a meal replacer, a pill or a tablet.

In one embodiment, the food composition according to the present invention is a dairy based product, for example a liquid dairy based product or a powdered dairy based product to be reconstituted in milk or water.

In one embodiment, particularly well accepted by consumers are powdered nutritional compositions to be reconstituted in milk or water.

In another embodiment, also well accepted are nutritional supplements, for example in the form of a tablet or a gummy.

In one embodiment, the food composition comprises a nutrients' mixture consisting of Vitamin A and Vitamin D only.

In one embodiment, the supplement provides selected nutrients while not representing a significant portion of the overall nutritional needs of the subject and/or does not represent more than 0.1%, 1%, 5%, 10%, or 20% of the daily energy need of the subject.

Vitamin A

In one embodiment of the present invention, a food composition is provided which comprises Vitamin A.

Vitamin A may be incorporated in the composition of the invention as such or in the form of a physiologically acceptable derivative such as a salt and/or via any source comprising Vitamin A. For example ingredients may be selected in the group consisting of: carrots, red sweet peppers, turnips, orange juice, oranges, tomatoes, dark green leafy vegetables (such as spinach, broccoli and kale), cantaloupe, oranges, tomatoes, apricot, plantains, mangos, passion fruits, squash, yellow corn, soybeans, pistachio nuts, egg yolk, butter, milk, liver, cod liver oil and mixtures thereof. Vitamin pre-mix for fortification purpose is also included.

In one embodiment, Vitamin A is provided as such to the food composition.

As it is evident to the person skilled in the art, different ingredients may provide different amounts of Vitamin A in the composition according to the present invention, depending on the nature and amount of the ingredient used. It will be nonetheless routine work to the skilled person to calculate the amount of ingredient needed to provide the claimed amount of Vitamin A, based on the specification of the specific ingredient provided by the supplier.

In one embodiment, the food composition of the present invention comprises Vitamin A in an amount of at least 350 μg per serving, for example at least 400 μg per serving. In another embodiment, the food composition of the present invention comprises Vitamin A in an amount ranging from 350 to 3000 μg per serving. In a further embodiment, the food composition of the present invention comprises Vitamin A in an amount ranging from 350 to 600 μg per serving. In such embodiment, the food composition of the present invention delivers the total daily amount of Vitamin A considered necessary to address the nutrient's deficiency in the identified group of lactating women.

In one embodiment, the food composition according to the present invention may be administered in 1, 2, 3 or 4 daily servings to provide the total daily amounts of Vitamin A as above described. In such embodiment, as it will be apparent to a person skilled in the art, the amount of Vitamin A contained in each serving of the food composition according to the present invention will be divided by 1, 2 3 or 4 respectively.

In one embodiment, the food composition according to the present invention is intended for consumption once or twice per day.

Vitamin D

In one embodiment of the present invention, a food composition is provided which comprises Vitamin D.

Vitamin D may be incorporated in the composition of the invention as such or in the form of a physiologically acceptable salt and/or via any source comprising Vitamin D. For example ingredients may be selected in the group consisting of: cow's milk, egg yolk, liver, fish liver oils, fatty fish, mushrooms and mixtures thereof. Vitamin pre-mix for fortification purpose is also included.

As it is evident to the person skilled in the art, different ingredients may provide different amounts of Vitamin D in the composition according to the present invention, depending on the nature and amount of the ingredient used. It will be nonetheless routine work to the skilled person to calculate the amount of ingredient needed to provide the claimed amount of Vitamin D, based on the specification of the specific ingredient provided by the supplier.

In one embodiment, the food composition of the present invention comprises Vitamin D in an amount of at least 9 μg per serving, for example at least 10 μg per serving. In another embodiment, the food composition of the present invention comprises Vitamin D in an amount ranging from 9 to 100 μg per serving. In a further embodiment, the food composition of the present invention comprises Vitamin D in an amount ranging from 9 to 20 μg per serving.

In such embodiment, the food composition of the present invention delivers the total daily amount of Vitamin D considered necessary to address the nutrient's deficiency in the identified group of lactating women.

In one embodiment, the food composition according to the present invention may be administered in 1, 2, 3 or 4 daily servings to provide the total daily amounts of Vitamin D as above described. In such embodiment, as it will be apparent to a person skilled in the art, the amount of Vitamin D contained in each serving of the food composition according to the present invention will be divided by 1, 2 3 or 4 respectively.

In one embodiment, the food composition according to the present invention is intended to be consumed once or twice per day.

Health Benefits

The present invention provides for a food composition for use in preventing nutrients' inadequacies in lactating women.

In one embodiment, the food composition according to the present invention reduces the risk of nutrients' inadequacies by helping to achieving nutrients' adequacy to support the nutritional needs of the lactating mother.

In another embodiment, the food composition according to the present invention reduces the risk of nutrients' inadequacies by helping to avoid nutrient depletion in the lactating mothers' stores and help transferring the nutrients to her baby without compromising her stores.

In one embodiment, the food composition of the present invention is intended for administration to a lactating woman for 24 or 18 months after she gave birth.

In another embodiment, the food composition of the present invention is intended for administration to a lactating woman for 12 months after she gave birth. In another embodiment, the food composition of the present invention is intended for administration to a lactating woman for 6 months after she gave birth.

In one aspect, the present invention provides for use of Vitamin A or Vitamin D in the manufacture of a food composition to prevent nutrients' inadequacies in lactating women having high fiber type dietary pattern.

### EXPERIMENTAL SECTION

### Example 1

Identification of Clusters of Women Based on Dietary Pattern Analysis

The data used for this analysis are from 6 European countries. Participants were eligible when they were between 18 and 40 years at the time of enrolment, had a BMI before pregnancy between 19 and 29 and had decided to exclusively breast-feed their new-born from birth up to 4 months of age.

Assessment of Dietary Intake

The dietary intake was estimated using 3-day food diaries and calculated as a 3-day average. Food diaries were collected at 6 time points post-partum up to 4 months of lactation. In detail, the participants were observed from the last visit immediately preceding their delivery (V0), and then observed for six follow-up visits after delivery, i.e. at day 2 (V1), 17 (V2), 30 (V3), 60 (V4), 90 (V5) and 120 (V6). At each visit, the participants were asked to provide information on their dietary consumption over the preceding three days. The participants' dietary information from all countries was then translated to consumption data, food group data and nutrient daily intake data using a French food group classification and nutrient composition database (CIQUAL).

Statistical Analysis

The analyses were performed on the 29 food group variables as per the Ciqual 2013 database for the set of 180 participants in the V2-V6 subset. V1 data were excluded because women were usually still in the hospital, which is not a reflection of the normal dietary habits. For each visit, nutrient intakes for each participant were calculated using food item record and food composition values of Ciqual 2017 database.

Based on Ciqual database 2013, the main food groups included in the analyses are: ‘Fruits’, ‘Legumes’, ‘Starchy_tubers’, ‘Nuts_and_seeds’, ‘Breads_and_rolls’, ‘Brk_cereals_and_cereal_bar’, ‘Cereals_and_pasta’, ‘Pastry_and_biscuits’, ‘Eggs_and_egg_products’, ‘Milk_and_milk_products’, ‘Cheeses’, ‘Mixed_salads’, ‘Mixed_dishes’, ‘Sandwiches’, ‘Fish_and_bat’, ‘Shellfish_and_mol’, ‘Meat_products’, ‘Poultry’, ‘Red_meat’, ‘Coffee_tea_cocoa_beverages’, ‘Soft_drinks’, ‘Juices_and_nectars’, ‘Fats_and_oils’, ‘Seasonings_and_sauces’, ‘Soups_and_stocks’. Some food groups were excluded, such as ‘Baby food’, ‘Dietary Supplements’, ‘Offals’, ‘Prd_for_special_nutritional_use’ because only a small number of women consumed significant amounts. To create the dataset for further analyses of dietary patterns, participants with outlier diets (less than 1074.8 kcal or more than 4776.9 kcal per day) were removed (Meltzer H M, Brantsaeter A L, Ydersbond T A et al. 2008, Methodological challenges when monitoring the diet of pregnant women in a large study: experiences from the Norwegian Mother and Child Cohort Study (MoBa). Matern Child Nutr 4, 14-27).

K-means clustering method, which is commonly used in dietary pattern analysis, was applied to identify dietary patterns from the early stage and up to 4 months of lactation. The analyses were performed for 3 to 5 clusters. The 4 cluster solution was chosen using the gap statistic approach, basically taking into consideration the amount of variation explained by the solution, the size and interpretability of each cluster, and the stability of the solution, which was evaluated using linear discriminant analysis.

Cluster analysis is the study of how to partition a set of items into non-overlapping groups, or clusters, such that items within a cluster are more similar to each other than to items in other clusters. As a result, four clusters were found.

Table 1 reports, in each of the clusters, the mean intake and standard deviation for all the analysed food groups based on Ciqual database food grouping of 2013:

| Food intake (grams/day) | Mean (std) of C1 | | Mean (std) of C2 | | Mean (std) of C3 | | Mean (std) of C4 | |
|---|---|---|---|---|---|---|---|---|
| Alc_beverages | 47.9 | (150.2) | 8.2 | (17.7) | 25.8 | (34.8) | 28.3 | (62.7) |
| Breads_and_rolls | 84.0 | (50.5) | 86.5 | (34.8) | 113.4 | (45.5) | 79.4 | (32.1) |
| Brk_cereals_and_cereal_bar | 34.2 | (78.7) | 8.8 | (14.2) | 19.3 | (31.6) | 17.4 | (27.7) |
| Cereals_and_pasta | 84.3 | (46.0) | 121.4 | (53.2) | 77.0 | (45.4) | 72.1 | (36.0) |
| Cheeses | 36.7 | (21.9) | 12.0 | (10.5) | 46.4 | (27.5) | 28.2 | (19.1) |
| Eggs_and_egg_products | 16.7 | (11.5) | 13.7 | (11.2) | 13.5 | (11.8) | 10.6 | (12.5) |
| Fats_and_oils | 20.2 | (9.7) | 9.3 | (4.6) | 12.8 | (9.5) | 10.7 | (6.7) |
| Fish_and_bat | 28.2 | (25.1) | 55.9 | (28.8) | 22.1 | (20.2) | 17.0 | (15.8) |
| Fruits | 226.9 | (127.0) | 211.9 | (115.1) | 150.7 | (106.4) | 116.2 | (78.4) |
| Juices_and_nectars | 71.4 | (62.0) | 56.2 | (60.6) | 74.1 | (73.7) | 112.7 | (100.0) |
| Legumes | 13.5 | (17.3) | 6.3 | (9.0) | 5.5 | (8.4) | 3.2 | (6.3) |
| Meat_products | 29.3 | (22.7) | 16.7 | (14.2) | 35.4 | (26.8) | 34.4 | (21.3) |
| Milk_and_milk_products | 264.2 | (152.8) | 361.6 | (175.7) | 235.0 | (131.8) | 257.0 | (182.2) |
| Mixed_dishes | 73.1 | (50.3) | 36.6 | (28.9) | 69.0 | (43.0) | 132.7 | (58.3) |
| Mixed_salads | 2.2 | (5.4) | 2.7 | (9.5) | 18.8 | (22.1) | 4.1 | (7.2) |
| Prepared_Non_Alc_beverages | 610.4 | (882.4) | 709.9 | (618.7) | 734.3 | (797.0) | 611.4 | (626.7) |
| Nuts_and_seeds | 10.1 | (13.9) | 1.6 | (4.2) | 3.4 | (4.3) | 2.9 | (5.4) |
| Pastry_and_biscuits | 46.7 | (29.3) | 44.4 | (28.0) | 46.6 | (22.5) | 75.1 | (39.2) |
| Poultry | 20.4 | (19.4) | 52.0 | (31.6) | 22.6 | (18.7) | 25.0 | (23.8) |
| Red_meat | 42.9 | (39.8) | 74.1 | (40.6) | 37.5 | (21.8) | 39.5 | (27.4) |
| Sandwiches | 0.9 | (3.9) | 3.6 | (6.5) | 6.6 | (12.8) | 16.4 | (19.6) |
| Seasonings_and_sauces | 22.4 | (19.3) | 6.0 | (6.9) | 18.2 | (16.0) | 14.4 | (9.6) |
| Shellfish_and_mol | 7.8 | (14.7) | 8.3 | (13.5) | 6.6 | (14.6) | 8.1 | (14.5) |
| Soups_and_stocks | 28.0 | (43.4) | 130.2 | (102.1) | 62.7 | (75.9) | 40.6 | (56.8) |
| Starchy_tubers | 55.4 | (49.2) | 90.4 | (40.7) | 52.2 | (30.5) | 51.9 | (36.7) |
| Coffee_tea_cocoa_beverages | 164.4 | (167.1) | 86.1 | (75.7) | 378.3 | (310.2) | 168.9 | (181.6) |
| Soft_drinks | 45.5 | (83.5) | 58.7 | (97.1) | 60.1 | (102.6) | 124.3 | (136.4) |
| Sugars_and_confectionery | 20.7 | (13.1) | 9.6 | (11.0) | 42.8 | (22.8) | 17.9 | (13.5) |
| Vegetables | 206.6 | (91.2) | 71.8 | (51.1) | 121.7 | (45.3) | 97.6 | (57.2) |

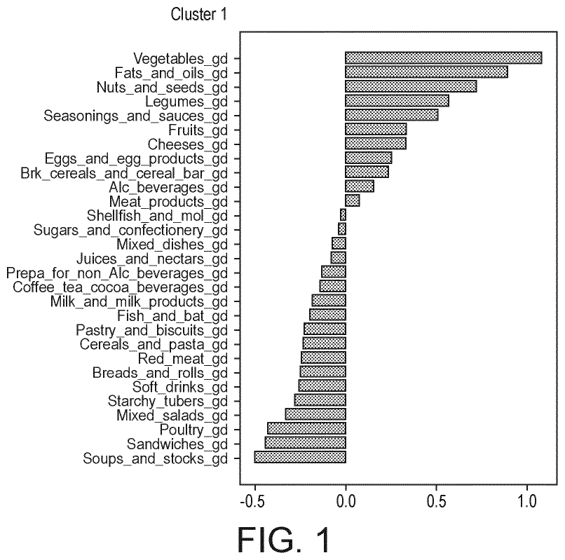

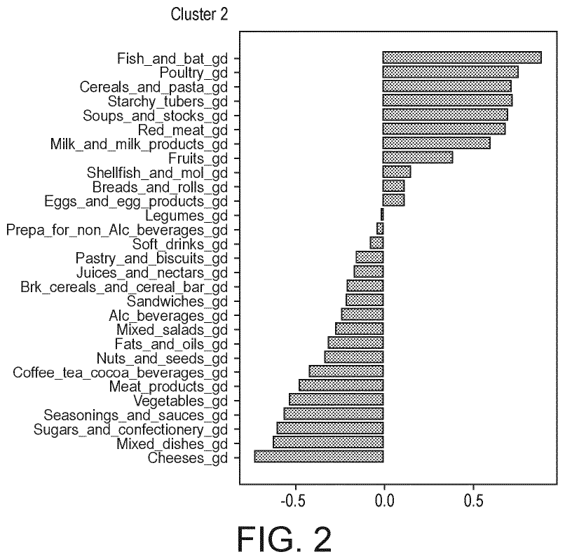

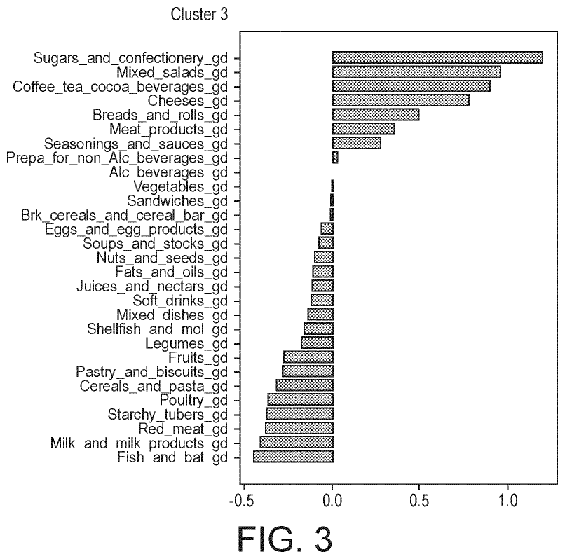

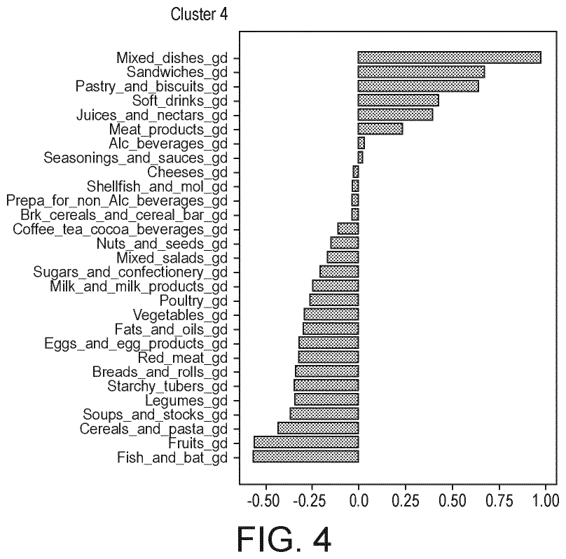

FIGS. 1 to 4 schematically represent the food intake features of each identified clusters (Clusters 1 to 4, respectively). Y-axis shows the food groups, x-axis shows the z score of the difference between individual intake in each group of people (i.e. each “cluster”) and the mean intake of total population.

The nutrient intake of each cluster was calculated. Table 1 below reports the nutrients' mean daily intake (Mean) and standard deviation (std) in each cluster.

TABLE 2

| | Mean (std) of C1 | | Mean (std) of C2 | | Mean (std) of C3 | | Mean (std) of C4 | |
|---|---|---|---|---|---|---|---|---|
| Energy (kcal) | 2112.7 | (441.3) | 1887.7 | (330.7) | 2114.5 | (399.5) | 2122.2 | (293.1) |
| Proteins (g) | 82.9 | (19.2) | 97.0 | (15.4) | 81.3 | (16.3) | 82.8 | (12.7) |
| Fat (g) | 86.0 | (20.9) | 59.7 | (13.4) | 88.2 | (20.1) | 85.6 | (15.1) |
| Carbohydrates (g) | 237.2 | (58.8) | 227.8 | (46.7) | 233.8 | (51.9) | 241.7 | (37.5) |
| Alcohol (g) | 1.2 | (2.0) | 0.5 | (1.1) | 1.4 | (2.1) | 1.3 | (2.5) |
| Starch total (g) | 113.4 | (37.7) | 119.6 | (24.8) | 111.8 | (30.9) | 115.9 | (22.9) |
| Dietary fibre (g) | 25.1 | (8.9) | 18.1 | (4.9) | 19.9 | (5.0) | 18.6 | (4.6) |
| Sugars (g) | 95.7 | (25.0) | 90.5 | (29.0) | 97.5 | (27.5) | 98.2 | (24.7) |
| Selenium (μg) | 144.9 | (60.3) | 173.1 | (54.1) | 128.5 | (59.0) | 112.8 | (31.5) |
| Iodine (μg) | 168.6 | (160.2) | 160.4 | (37.5) | 159.2 | (152.3) | 138.5 | (51.1) |
| Potassium (mg) | 3373.6 | (1345.6) | 3088.3 | (610.8) | 2852.5 | (652.2) | 2852.4 | (723.2) |
| Phosphorus (mg) | 1390.9 | (343.2) | 1328.8 | (241.1) | 1339.5 | (305.6) | 1308.7 | (263.8) |
| Iron (mg) | 16.3 | (18.1) | 8.6 | (1.9) | 33.1 | (120.6) | 12.3 | (8.4) |
| Manganese (mg) | 4.8 | (4.2) | 2.3 | (0.6) | 3.4 | (1.2) | 3.6 | (4.1) |
| Copper (mg) | 1.8 | (1.1) | 1.2 | (0.3) | 1.8 | (1.0) | 1.5 | (0.5) |
| Calcium (mg) | 1077.3 | (416.1) | 813.0 | (287.9) | 1036.7 | (419.8) | 967.1 | (310.4) |
| Sodium (mg) | 2601.1 | (652.9) | 2239.4 | (585.0) | 2829.6 | (559.5) | 3255.5 | (1668.3) |
| Salt (g) | 3.9 | (1.6) | 3.9 | (1.2) | 3.6 | (1.1) | 3.7 | (1.4) |
| Magnesium (mg) | 380.0 | (154.0) | 288.9 | (59.3) | 341.4 | (144.1) | 298.6 | (67.8) |
| Zinc (mg) | 11.5 | (8.1) | 9.3 | (1.8) | 10.9 | (7.9) | 9.7 | (2.2) |
| Vitamin C (mg) | 134.4 | (115.7) | 108.3 | (49.3) | 126.7 | (132.3) | 118.4 | (69.0) |
| Niacin (mg) | 21.9 | (16.8) | 23.0 | (4.7) | 20.1 | (17.1) | 19.5 | (6.3) |
| Vitamin B6 (mg) | 2.2 | (1.4) | 2.0 | (0.4) | 2.0 | (1.5) | 1.8 | (0.6) |
| Vitamin B12 (μg) | 5.9 | (3.2) | 7.7 | (4.1) | 5.8 | (4.2) | 6.9 | (7.2) |
| Pantothenic acid (mg) | 5.9 | (4.7) | 5.4 | (1.1) | 5.9 | (4.3) | 5.2 | (2.0) |
| Folate, total (μg) | 446.2 | (434.2) | 257.2 | (62.9) | 378.4 | (435.5) | 306.9 | (89.9) |
| Riboflavin (mg) | 2.0 | (1.7) | 1.7 | (0.4) | 2.0 | (1.6) | 1.8 | (0.6) |
| Thiamin (mg) | 2.1 | (2.4) | 1.3 | (0.3) | 1.5 | (1.5) | 1.5 | (0.6) |
| Vitamin D (μg) | 6.2 | (8.9) | 5.0 | (1.8) | 5.6 | (9.2) | 4.5 | (4.5) |
| Vitamin E (mg) | 16.7 | (24.6) | 7.2 | (2.3) | 14.8 | (25.4) | 11.4 | (5.1) |
| Retinol (μg) | 398.4 | (287.5) | 247.7 | (96.5) | 496.0 | (324.0) | 494.0 | (463.9) |
| Vitamin A (μg_RE) | 940.2 | (453.8) | 684.3 | (266.1) | 930.3 | (358.0) | 788.0 | (435.8) |
| Beta-carotene (μg) | 3691.2 | (2285.9) | 2657.0 | (1507.7) | 3080.9 | (2224.1) | 2094.7 | (1009.8) |
| Fatty acid 4:0 (g) | 0.8 | (0.3) | 0.6 | (0.2) | 1.2 | (0.5) | 1.0 | (0.4) |
| Fatty acid 6:0 (g) | 0.6 | (0.2) | 0.5 | (0.2) | 0.8 | (0.3) | 0.8 | (0.4) |
| Fatty acid 8:0 (g) | 0.5 | (0.2) | 0.4 | (0.1) | 0.6 | (0.2) | 0.6 | (0.3) |
| Fatty acid 10:0 (g) | 0.8 | (0.3) | 0.6 | (0.2) | 1.1 | (0.4) | 1.0 | (0.5) |
| Fatty acid 12:0 (g) | 1.7 | (1.0) | 1.0 | (0.4) | 1.8 | (0.6) | 2.0 | (1.2) |
| Fatty acid 14:0 (g) | 3.2 | (1.2) | 2.3 | (0.7) | 3.9 | (1.5) | 3.4 | (1.2) |
| Fatty acid 16:0 (g) | 15.0 | (4.1) | 12.1 | (2.8) | 17.5 | (5.0) | 16.2 | (4.3) |
| Fatty acid 18:0 (g) | 5.9 | (2.1) | 4.7 | (1.4) | 7.3 | (2.2) | 6.5 | (1.9) |
| Fatty acid 18:1 cis | 27.1 | (8.3) | 16.3 | (4.6) | 23.6 | (5.9) | 23.3 | (4.7) |
| omega-9 (g) | | | | | | | | |
| Fatty acid cis 18:2 | 8.1 | (3.3) | 4.6 | (1.3) | 6.8 | (2.4) | 6.8 | (1.9) |
| omega-6 (g) | | | | | | | | |
| Fatty acid 18:3 omega-3 | 1.3 | (0.8) | 0.7 | (0.2) | 0.9 | (0.3) | 1.0 | (0.3) |
| (ALA) (g) | | | | | | | | |
| Fatty acid 20:4 omega-6 (g) | 0.1 | (0.0) | 0.1 | (0.0) | 0.1 | (0.0) | 0.1 | (0.0) |
| Fatty acid 20:5 omega-3 | 0.2 | (0.1) | 0.2 | (0.2) | 0.2 | (0.2) | 0.1 | (0.1) |
| (EPA) (g) | | | | | | | | |
| Fatty acid 22:6 omega-3 | 0.2 | (0.2) | 0.4 | (0.2) | 0.2 | (0.2) | 0.2 | (0.2) |
| (DHA) (g) | | | | | | | | |
| saturated (SFA) (g) | 33.0 | (9.2) | 24.3 | (6.0) | 37.7 | (11.0) | 35.6 | (9.1) |
| monounsaturated (g) | 31.9 | (8.8) | 19.6 | (5.3) | 29.1 | (6.9) | 28.6 | (5.1) |
| polyunsaturated (g) | 11.7 | (4.5) | 7.1 | (1.9) | 10.5 | (3.3) | 10.3 | (2.7) |
| Cholesterol (mg) | 276.5 | (76.5) | 318.1 | (61.3) | 319.8 | (121.4) | 298.3 | (66.1) |
| Polyols (g) | 1.6 | (1.5) | 1.6 | (1.2) | 0.9 | (1.0) | 1.0 | (1.1) |
| Organic acids total (g) | 4.2 | (1.5) | 3.9 | (1.8) | 3.5 | (1.6) | 3.5 | (1.4) |

### Example 2

Identification of Nutrients' Inadequacies in the Clusters

The nutrients' daily dietary intake of lactating women belonging to the identified clusters were confronted with EFSA dietary recommendations for lactating women. As a result, nutrients inadequacies were identified for each cluster. Results are reported in table 3 below.

TABLE 3

| | | | | | | | | Difference between clusters | | | |
|---|---|---|---|---|---|---|---|---|---|---|---|
| | | | | | | | | and EFSA recommendation | | | |
| EFSA recommendation | | | | Mean | Mean | Mean | Mean | for lactating women | | | |
| Nutrient | Unit | PRI | Energy/Nutrient | of C1 | of C2 | of C3 | of C4 | C1 | C2 | C3 | C4 |
| Fibre | g | 25 | Fibre (g) | 25.1 | 18.1 | 19.9 | 18.6 | 0.1 | −6.9 | −5.1 | −6.4 |
| Sugars | % E | 10 | Sugars (g) | 95.7 | 90.5 | 97.5 | 98.2 | | | | |
| Selenium | ug | 85 | Selenium (μg) | 144.9 | 173.1 | 128.5 | 112.8 | 59.9 | 88.1 | 43.5 | 27.8 |
| Iodine | ug | 200 | Iodine (μg) | 168.6 | 160.4 | 159.2 | 138.5 | −31.4 | −39.6 | −40.8 | −61.5 |
| Potassium | mg | 4000 | Potassium (mg) | 3373.6 | 3088.3 | 2852.5 | 2852.4 | −626.4 | −911.7 | −1147.5 | −1147.6 |
| Phosphorus | mg | 550 | Phosphorus (mg) | 1390.9 | 1328.8 | 1339.5 | 1308.7 | 840.9 | 778.8 | 789.5 | 758.7 |
| Iron | mg | 16 | Iron (mg) | 16.3 | 8.6 | 33.1 | 12.3 | 0.3 | −7.4 | 17.1 | −3.7 |
| Manganese | mg | 3 | Manganese (mg) | 4.8 | 2.3 | 3.4 | 3.6 | 1.8 | −0.7 | 0.4 | 0.6 |
| Copper | mg | 1.5 | Copper (mg) | 1.8 | 1.2 | 1.8 | 1.5 | 0.3 | −0.3 | 0.3 | 0.0 |
| Calcium | mg | 1000 | Calcium (mg) | 1077.3 | 813 | 1036.7 | 967.1 | 77.3 | −187.0 | 36.7 | −32.9 |
| Sodium | mg | 2000 | Sodium (mg) | 2601.1 | 2239.4 | 2829.6 | 3255.5 | 601.1 | 239.4 | 829.6 | 1255.5 |
| Magnesium | mg | 300 | Magnesium (mg) | 380 | 288.9 | 341.4 | 298.6 | 80.0 | −11.1 | 41.4 | −1.4 |
| Zinc | mg | 12.2 | Zinc (mg) | 11.5 | 9.3 | 10.9 | 9.7 | −0.7 | −2.9 | −1.3 | −2.5 |
| Vitamin C | mg | 155 | Vitamin C (mg) | 134.4 | 108.3 | 126.7 | 118.4 | −20.6 | −46.7 | −28.3 | −36.6 |
| Niacin | mg/MJ | 1.6 | Niacin (mg) | 2.48 | 2.91 | 2.27 | 2.20 | 0.88 | 1.31 | 0.67 | 0.60 |
| Vitamin B6 | mg | 1.7 | Vitamin B6 (mg) | 2.2 | 2 | 2 | 1.8 | 0.5 | 0.3 | 0.3 | 0.1 |
| Vitamin B12 | ug | 5 | Vitamin B12 (μg) | 5.9 | 7.7 | 5.8 | 6.9 | 0.9 | 2.7 | 0.8 | 1.9 |
| Pantothenic acid | mg | 7 | Pantothenic acid (mg) | 5.9 | 5.4 | 5.9 | 5.2 | −1.1 | −1.6 | −1.1 | −1.8 |
| Folate | ug | 500 | Folate, total (μg) | 446.2 | 257.2 | 378.4 | 306.9 | −53.8 | −242.8 | −121.6 | −193.1 |
| Riboflavin | mg | 2 | Riboflavin (mg) | 2 | 1.7 | 2 | 1.8 | 0.0 | −0.3 | 0.0 | −0.2 |
| Thiamin | mg/MJ | 0.1 | Thiamin (mg) | 0.24 | 0.16 | 0.17 | 0.17 | 0.14 | 0.06 | 0.07 | 0.07 |
| Vitamin D | ug | 15 | Vitamin D (μg) | 6.2 | 5 | 5.6 | 4.5 | −8.8 | −10.0 | −9.4 | −10.5 |
| Vitamin E (US), | mg | 19 | Vitamin E (mg) | 16.7 | 7.2 | 14.8 | 11.4 | −2.3 | −11.8 | −4.2 | −7.6 |
| RDA | | | | | | | | | | | |
| Vitamin A | ug | 1300 | Vitamin A (μg_RE) | 940.2 | 684.3 | 930.3 | 788 | −359.8 | −615.7 | −369.7 | −512.0 |
| Linoleic acid (LA) | E % | 4 | Fatty acid cis,cis | 8.1 | 4.6 | 6.8 | 6.8 | | | | |
| | | | 18:2 omega-6 (g) | | | | | | | | |
| Alpha-linolenic acid | E % | 0.5 | Fatty acid 18:3 | 0.55 | 0.33 | 0.38 | 0.42 | 0.1 | −0.2 | −0.1 | −0.1 |
| (ALA) | | | omega-3 (ALA) (g) | | | | | | | | |
| EPA + DHA | mg | 450 | EPA + DHA | 370 | 587 | 361 | 282 | −80.0 | 137.0 | −89.0 | −168.0 |
| Saturated Fat (SFA) | % E | Max. 10 | Fatty acids, | 33 | 24.3 | 37.7 | 35.6 | | | | |
| | | | saturated (SFA) (g) | | | | | | | | |

For cluster 1 for example, which is also referred to as the cluster of lactating women having a high fiber type dietary pattern, an inadequacy in the intake of Vitamin A and Vitamin D was identified.

### Example 3

Dietary Supplement Composition

A food compositions in the form of a dietary supplement according to the present invention may be prepared by incorporating into a capsule ingredients comprising the following nutrients:

Vitamin A 400 μg

Vitamin D 10 μg

The product is intended for daily consumption (one serving per day) by a lactating woman having a high fiber type dietary pattern and may prevent nutrients' inadequacies occurring in lactating woman having such a type of dietary intake.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

1. Method for use in preventing nutrients' inadequacies in lactating women having high fiber type dietary pattern comprising administering a food composition comprising at least one ingredient selected in the group consisting of:
Vitamin A; and
Vitamin D to a woman in need of same.

2. Method according to claim 1 wherein the food composition comprises at least one ingredient selected from the group consisting of:
Vitamin A in an amount of at least 350 mg per serving; and
Vitamin D in an amount of at least 9 mg per serving;
wherein the food composition is for a single daily administration.

3. Method according to claim 1 wherein the food composition comprises at least one ingredient selected from the group consisting of:
Vitamin A in an amount ranging from 350 mg to 3000 mg per serving; and
Vitamin D in an amount ranging from 9 mg to 100 mg per serving;
wherein the food composition is for a single daily administration.

4. Method according to claim 1 wherein the food composition comprises Vitamin A and Vitamin D.

5. Method according to claim 4 wherein the food composition comprises
Vitamin A in an amount ranging from 350 mg to 600 mg per serving; and
Vitamin D in an amount ranging from 9 mg to 20 mg per serving;
wherein the food composition is for a single daily administration.

6. Food composition comprising:
Vitamin A in an amount of at least 350 mg per serving;
Vitamin D in an amount of at least 9 mg per serving;
wherein the food composition is for a single daily administration.

7. Food composition according to claim 6 which comprises:
Vitamin A in an amount ranging from 350 mg to 3000 mg per serving;
Vitamin D in an amount ranging from 9 mg to 100 mg per serving.

8. Food composition according to claim 6 which comprises a nutrients' mixture consisting of Vitamin A and Vitamin D.

9-11. (canceled)
